(19) United States
(12) Patent Application Publication
(10) Pub. No.: US 2004/0102783 A1
(43) Pub. Date: May 27, 2004

(54) Powered Kerrison-like Rongeur system

(76) Inventors: Chester E. Sutterlin III, Gainesville, FL (US); Nikolaus Gravenstein, Gainesville, FL (US); Eddy H. Del Rio, Royal Palm Beach, FL (US); Jose M. Lamanna, Jupiter, FL (US)

Correspondence Address: Norman Friedland, 11300 US Highway One, Suite 400, North Palm Beach, FL, 33408, US

(21) Appl. No.: 10/306,266

(22) Filed: Nov. 27, 2002

(51) Int. Cl. A61B 17/16

(52) U.S. Cl. 606/80; 606/83

(57) ABSTRACT

A motorized drill is mounted in the handle of a Kerrison Rongeur System utilized by a surgeon to perform surgical procedures on a patient, includes mechanism for moving the rotating drill toward the fixed upstanding foot mounted at the remote end of the pistol-shaped handle for removing bone which is located between the upstanding foot and the drill bit. In another embodiment, the drill is removably fixed in the handle and the foot moves relative to the drill bit. In either embodiment a trigger operated by the surgeon actuates the foot/drill and the trigger is biased to maximize the space between the foot and the drill bit. The foot shape and dimension can be modified for perfecting given surgical procedures. In one embodiment the drill motor releasing mechanism includes a pinion gear and rack performing the additional function of releasing the motor locked in position in the handle.

### TECHNICAL FIELD

This invention relates to Kerrison-like Rongeur systems and particularly to a Kerrison-like Rongeur system that has been modified to include a powered cutting end (hereinafter referred to as a PKS system).

### BACKGROUND OF THE INVENTION

As is well known in medical technology, the Kerrison Rongeur system is utilized in certain types of spinal operations where it is necessary to cut bone near the spinal column. For example, in a laminectomy procedure it may be necessary to trim away some of the bone that may be in close proximity to the nerve passing through the spinal canal and the Kerrison Rongeur system includes an elongated scissor type of instrument that includes a pair of small end plates that are spaced from each other and movable relative to the other, and a trigger-like device that moves one of the end plates relative to the other. The manual force exerted by the operator operating the trigger moving the plates together bites the boney structure for removing the same. Obviously, the end plates are relatively large in order to have sufficient strength to manifest the biting operation. Also, the shape of the end plate, that is that portion located at the very end of the instrument needs to be blunt. The shape and size of the end (often referred to as the foot plate) is predicated on the brute force that must be generated to do the bone biting and the foot plate must generate a counter force that matches the force of the other end plate that is moved relative thereto. An analogy to this operation is a pair of scissors that have cutting blades that are moved relative to each other. Obviously, the size of the scissors is predicated on the resistance exerted on the cutting blades. The more resistance offered to the cutting blade, the larger the required scissor. A scissor designed to cut sewing thread would be much smaller than a scissor designed to cut through cardboard and similarly, a scissor for cutting floor tile would presumably be larger than a scissor for cutting cardboard. Also, the force required by the operator would be different in each circumstance and the harder the material being cut would presumably require more force supplied by the operator.

We have found that we can obviate the force requirement that is necessary in use for surgical operations for cutting or chipping bone that has been heretofore done by a Kerrison Rongeur or other bone and tissue biting instrument by designing the instrument with a powered operated rotating cutter that is either moveable relative to an end mounted foot plate or vice versa, that is, moving the end mounted foot plate relative to the rotating cutter. This inventive instrument affords a number of advantages over the heretofore known Kerrison Rongeur System which are listed immediately hereinbelow. These advantages merely give examples and are not to be considered limitations to the scope of this invention.

1) The foot plate and cutter are smaller and the outside diameter at the working end is smaller allowing the surgeon to reach areas of the anatomy of the patient that were not heretofore reachable or as easily reachable;

2) The leading edge of the foot plate can be contoured so that it can be used to free tissue from the bone, enhancing the use of the PKS system;

3) Since the motor driving the cutter absorbs most of the load, the work required by the surgeon is much reduced; and

4) By having the cutter movable relative to the foot plate, the surgeon increases his feel as to the positioning of the cutter relative to the bone and hence, the hands-on feel of the surgeon is improved.

This invention contemplates the use of a pinion and rack gear system for longitudinal movement of the motor driving the cutter in one of the embodiments and the pinion gear is judiciously mounted so that it also functions as a pivot for the ingress and egress of the motor from the main body of the PKS system.

### SUMMARY OF THE INVENTION

An object of this invention is to provide an improved Kerrison Rongeur system for biting bone and tissue of a patient.

A feature of this invention is to provide a motor driven cutter in the PKS system that is movable relative to the foot plate. An alternative embodiment of this invention is a fixed motor/cutter and a movable foot plate.

Another feature of this invention for a PKS system that positions the cutter relative to the foot plate, is that the pinion gear that functions to move complementary rack gears also functions as a pivot for removing and inserting the motor into the PKS system.

Another feature of this invention is to provide a PKS system having a rotating cutter that is designed with a smaller overall outside diameter than heretofore known Kerrison Rongeur systems.

Another feature of this invention is to provide a powered PKS system with a contoured foot plate having the leading edge designed to participate in the surgical procedure.

A still further feature of this invention is to provide a powered PKS system that alleviates the force requirements of the surgeon and/or affords an enhanced feel to the surgeon.

The foregoing and other features of the present invention will become more apparent from the following description and accompanying drawings.

### BRIEF DESCRIPTION OF THE DRAWINGS

FIG. 1 is an exploded view in elevation illustrating the major components of the PKS system with a movable motor of this invention;

FIG. 2 is an exploded view in perspective illustrating the details of the mechanism for positioning the motor shown in the embodiment of depicted in FIG. 1;

FIG. 3 is a perspective view of the handle of the embodiment depicted in FIG. 1;

FIG. 4 is a view partly in section and partly full illustrating the PKS system of this invention depicted in FIG. 1;

FIG. 5 is a side view in elevation showing the assembled PKS system depicted in FIG. 1;

FIG. 6 is the handle and the mechanism for positioning the motor of the PKS system depicted in FIG. 1 illustrating a first position;

FIG. 6A is the same Fig. as depicted in FIG. 6 where the trigger is positioned to a second position;

FIG. 7 is a sectional view of another embodiment of this invention where the trigger moves the foot plate relative to the motor and drill bit; and

FIG. 8 is a top plan view of the embodiment depicted in FIG. 7.

These figures merely serve to further clarify and illustrate the present invention and are not intended to limit the scope thereof.

### DETAILED DESCRIPTION OF THE INVENTION

This invention in its preferred embodiment utilizes an air driven motor for powering the cutter and it should be obvious to anyone skilled in this technology that any surgical motor using various mediums can be utilized to power the cutter. Moreover, the invention is being described utilizing Micro Max®, Black Max® and eMax™ motors which are products available from the assignee of this patent application, and it should be understood that other types of pneumatic motors could be substituted therefor. In addition to the pneumatic powered motors, motors powered by different mediums could also be utilized in this invention. Suffice it to say that any motor that is capable of being inserted into and operable with the PKS system of this invention could be employed and that the scope of this invention is not limited to the motor disclosed in the preferred embodiment.

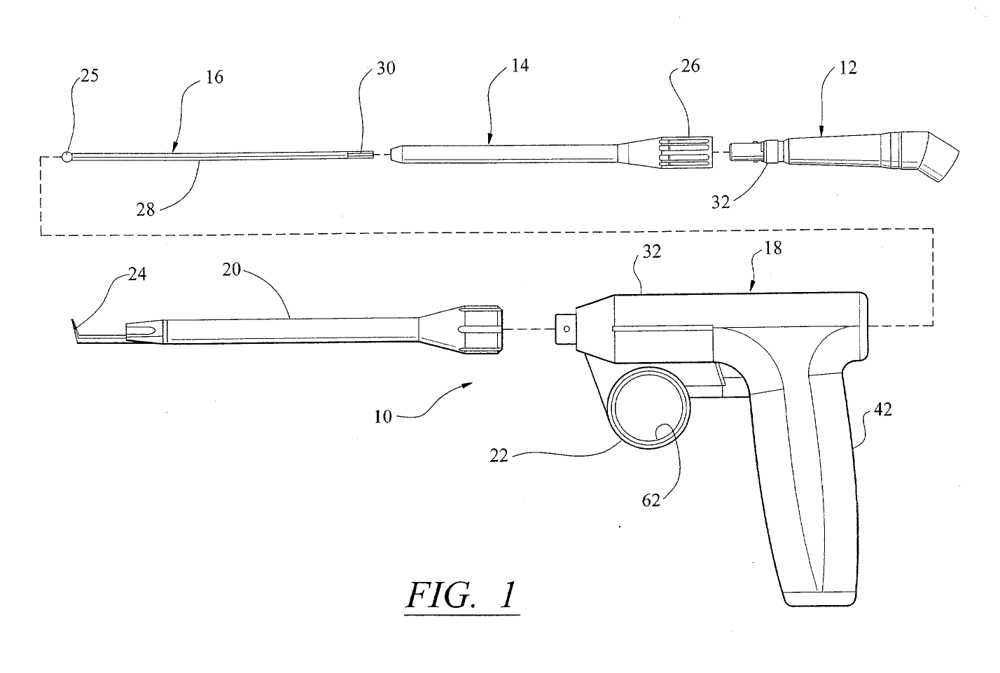

This portion of the description is directed to the embodiment of the PKS system where the motor/cutter is positioned relative to the foot plate and the following portion of the description will be directed to the embodiment of the PKS system where the foot plate is moved relative to the motor/cutter. As best seen in FIG. 1 the PKS system for the rectilinear movement of the motor/cutter is best illustrated in FIG. 1 which is an exploded view showing the PKS system generally illustrated by reference numeral 10 comprised of the motor 12, nose sleeve 14, cutter 16, pistol-shaped member or gun 18 and foot plate support 20. The foot plate support is an elongated tubular member that fits over the nose sleeve 14 when assembled. The motor 12, which in this embodiment, is a pneumatic motor, the nose sleeve 14 and the cutter 16 are well known and commercially available from the assignee. The gun 18 is shaped as a pistol handle for ease of operation but any other shape is considered to be within the scope of this invention and should afford a good feel to the surgeon when performing the medical procedure. As will be described in more detail hereinbelow, a trigger 22 is slidably mounted in the gun 18 and rectilinearly positions the motor 12. The foot plate support 20 is suitably removably attached to the end of gun 18 by any well known chuck or attachment mechanism. A foot plate 24 is carried at the end of foot plate support 20 and is angularly disposed relative to the axial axis of the foot plate support 20 and extends so as to be oriented in-line with the end of cutter 16. Hence, when assembled, the drill bit 24 of cutter 16 is in-line with the foot plate 24 and by virtue of being attached to motor 12 moves relative thereto when the trigger positions the motor rectilinearly. Because of this invention the foot plate 24 can be designed to accommodate surgical procedure requiring different sizes and shapes of the foot-plate. For example, the angle relative to the axial axis of the foot plate support 20 can be 30 degrees (°), 45°, etc.

The assembly of the PKS system is as follows, noting all the subassemblies can be readily removed and replaced. The nose sleeve 14 is suitably attached to the end of the motor 12 by any commercially available attaching mechanism 26. The shaft 28 of cutter 16 slides into the nose sleeve 14 and is supported for rotary motion by bearings (not shown) and includes an attachment end 30 that fits into a commercially available chuck 32 (not shown) carried by motor 12. As mentioned in the above paragraphs these components are commercially available from the assignee of this patent application.

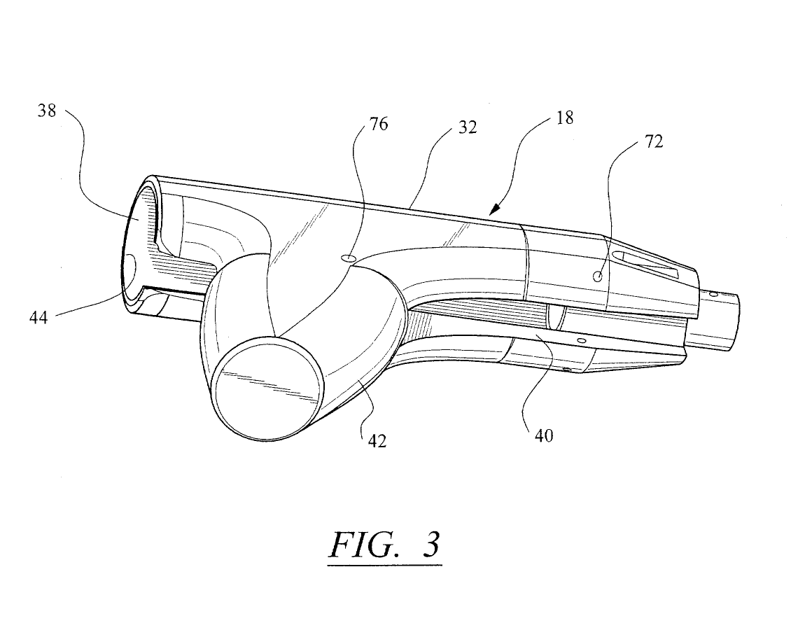

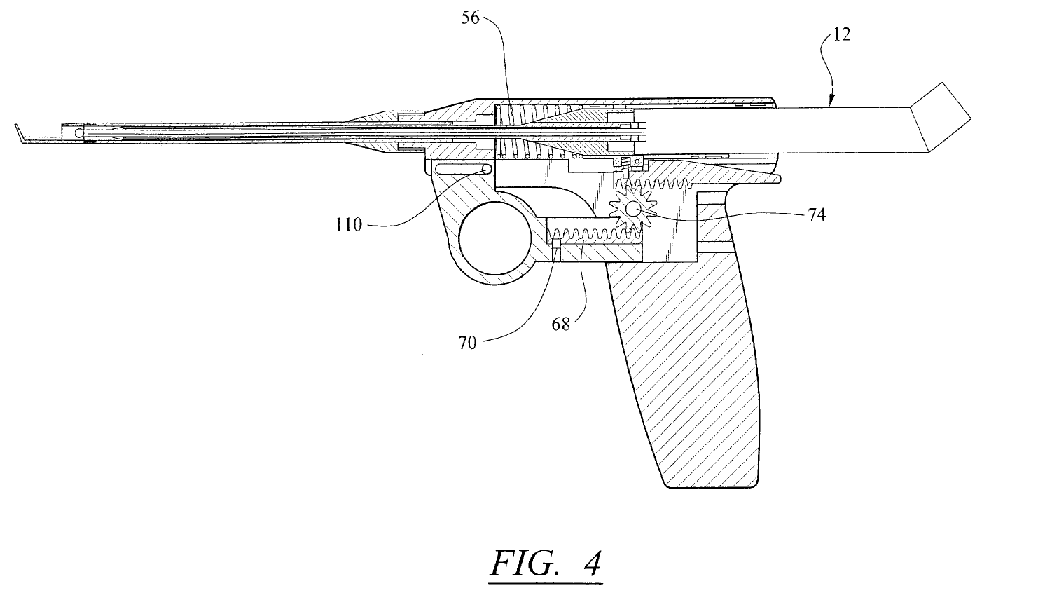

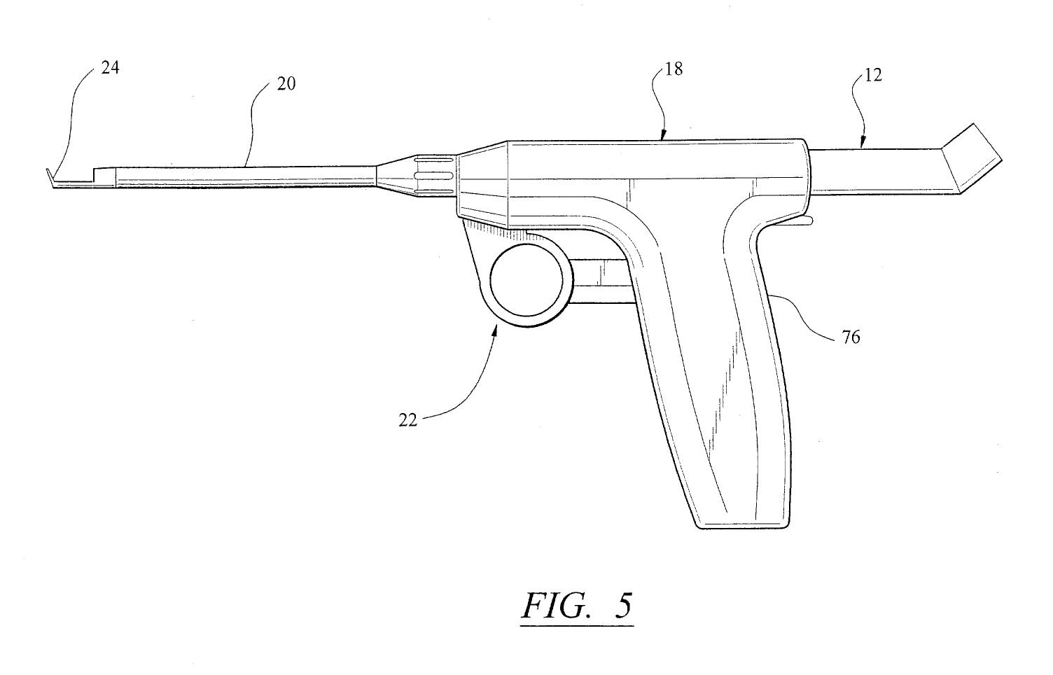

The motor 12, nose sleeve 14 and cutter 16 fit into an axial bore extending through the main body 32 of handle 18 and the nose sleeve 14 extends through the handle 18 so that the central passage extending through foot support 20 slides over the nose sleeve 14 and locates the bit 24 adjacent to the foot plate 22. The handle 18 (FIG. 3) includes a through axial bore 38 in the main body 40 extending on the underneath side of the main body 32 and into the hand held portion 42 of the handle 18 for receiving the trigger 22. From the foregoing it is apparent that the motor fits into the wider portion 44 of the bore 38 and the chuck portion 26 of the nose sleeve 14 fits into the smaller diameter portion 46 of the bore 38 and the movable trigger 22 fits into the slot 40. FIGS. 4 and 5 illustrate the PKS system in the assembled condition.

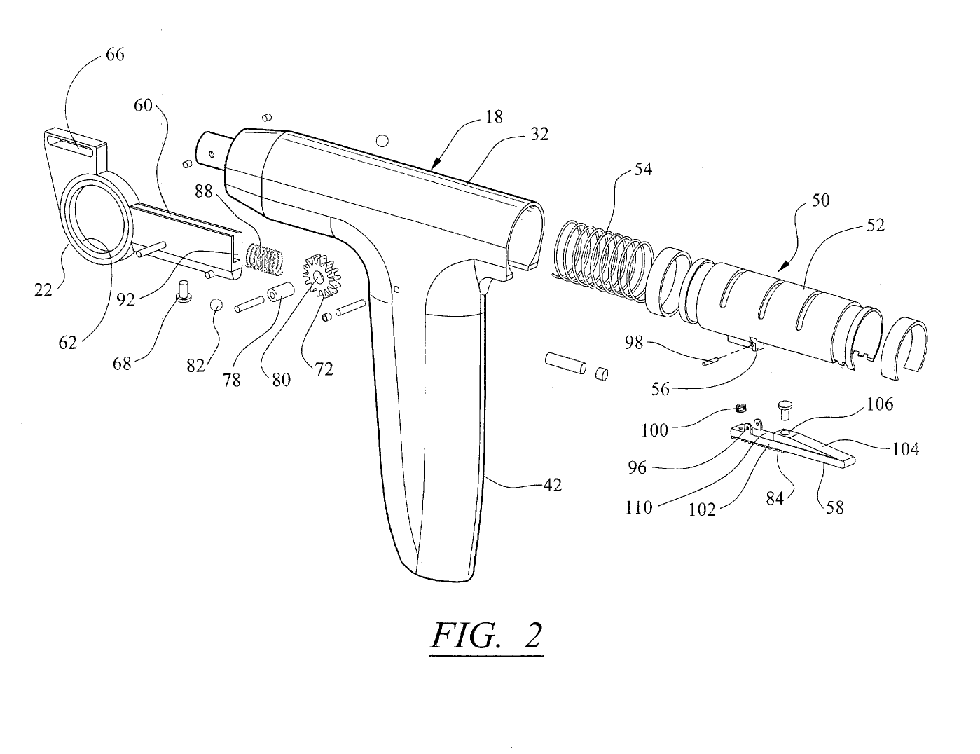

The next portion of the disclosure describes the mechanism for the trigger for rectilinearly positioning the motor and locking the motor into place. As best seen in FIGS. 2, 4, 6 & 6A, the motor support mechanism 50 includes a guide sleeve 52 mounted in the wider diameter portion 44 of the axial bore 38 and is biased on one end by coil spring 54 bearing against the shoulder 56 at the end of the wider diameter portion 44 and the front bushing 60 mounted adjacent to the end of guide sleeve 52. A back bushing 61 is mounted at the rear end of the guide sleeve 52 and together with the front bushing 60 support the motor when inserted into the bore 44. A boss 56 carried on the bottom of the guide sleeve 52 supports the motor lock 58 which will be described in further detail hereinbelow.

As mentioned above the trigger 22 is slidably mounted in slot 40 and includes a generally U-shaped portion or channel 60, a circular finger opening 62, an upper extending portion 64 and the limit slot 66. A rack gear 68 with the gear teeth facing upwardly is mounted in the channel 60 and rigidly supported therein by a pin 70. The pinion gear 72 is rotatory supported by axle or pin 74 that fits through diametrically opposing apertures 76 formed in main portion 32 of pistol 18. A bushing 78 fits into the central bore 80 of pinion gear 72 for ease of rotation. A spherical ball 82 fits into a slot formed on the bottom of the channel 60 for ease of movement of the trigger 22. The complementary rack gear 84 is carried on the bottom surface of the motor lock 58 with the gear teeth diametrically opposing the gear teeth of the rack gear 68. Coil spring 88 is sandwiched between the shoulder 90 and the rear edge 92 of the channel portion 60. The coil spring 88, which serves to return trigger 22 to the starting position (when the cutter bit is furthest from the foot plate) in conjunction with the coil spring 54 acting on the end of guide sleeve 52 which biases the motor to the starting position, affords a balanced force on the PKS system to assure that the operator or surgeon has a good feel as to the handling of the gun and positioning of the cutter.

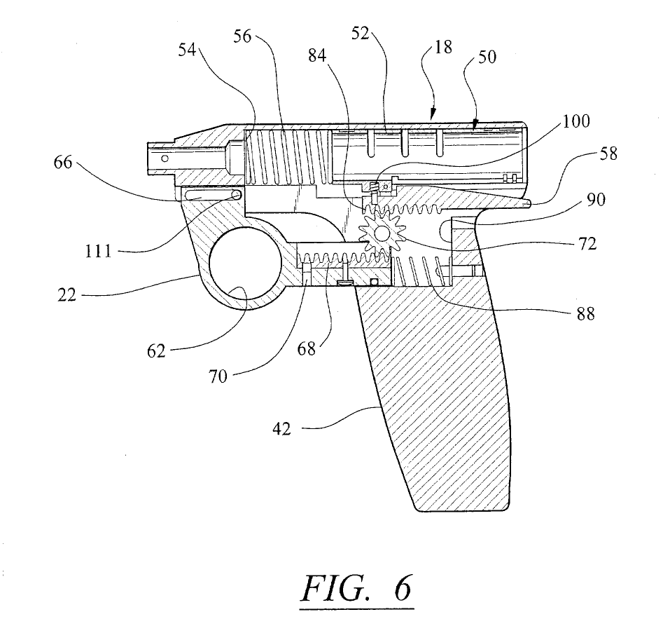

As mentioned above the motor lock 58 serves to lock the motor into place and is designed so that it automatically locks the motor when it is inserted into bore 44 and is released by depressing the end of the motor lock 58 when the trigger is in a given position. This assures that the motor will not disengage when the surgeon is performing the surgical procedure. In accordance with this invention, the motor lock 58 is mounted to the boss 56 via the upstanding parallel arms 96 and by the dowel pin 98. Coil spring 100 is fitted into a drilled hole formed on the bottom of boss 56 and bears against the top surface 102 of motor lock 58 at one end of the pivot remote from the front end. Ramp 104 is mounted on the aft end of motor lock 58 and fares from the thin portion at the aft end to a thicker portion forming a ramp so that when the motor is inserted and moved inwardly toward the front end of the gun a flange on the gun runs up the slope of the ramp and forces the motor lock 58 to pivot about pinion gear 72 and compresses spring 100 until the shoulder on the motor passes shoulder 106 at the end of ramp 104 on the motor lock 58 and drops into the recess 110 between the arms 96 and the end of the fore end of the ramp 104. The spring will force the motor lock to return to the original position locking the motor into place. It will be noted and in accordance with this invention, the motor lock 58 rotates about the pinion gear 72, i. e. the pinion gear 72 serves the dual function of the gear and pivot. Hence, when the trigger is withdrawn, namely when it places the cutter toward the foot plate and the stop pin 111 is at the end of the slot 66, the motor lock 58 cannot pivot and. (See FIG. A). The only time that the motor can be removed is when the pinion gear 72 is closer to the fore end of rack gear 84 so that it can pivot about the pinion gear 72 as shown in FIG. 6.

To operate the PKS system, the surgeon merely has to insert the foot plate in the position necessary to remove the bone from the patient, power the drill motor to rotate the cutter and squeeze the handle to position the cutter toward the foot plate. Obviously, the surgeon can move the PKS System when the cutter is exposed to the bone for not only localized cutting, but can also slide the cutter to follow a given path, if so desired, and cut the bone along a longitudinal path.

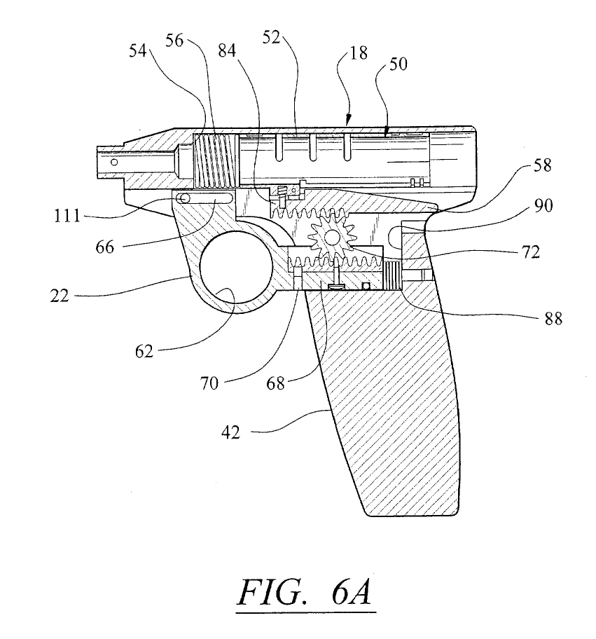

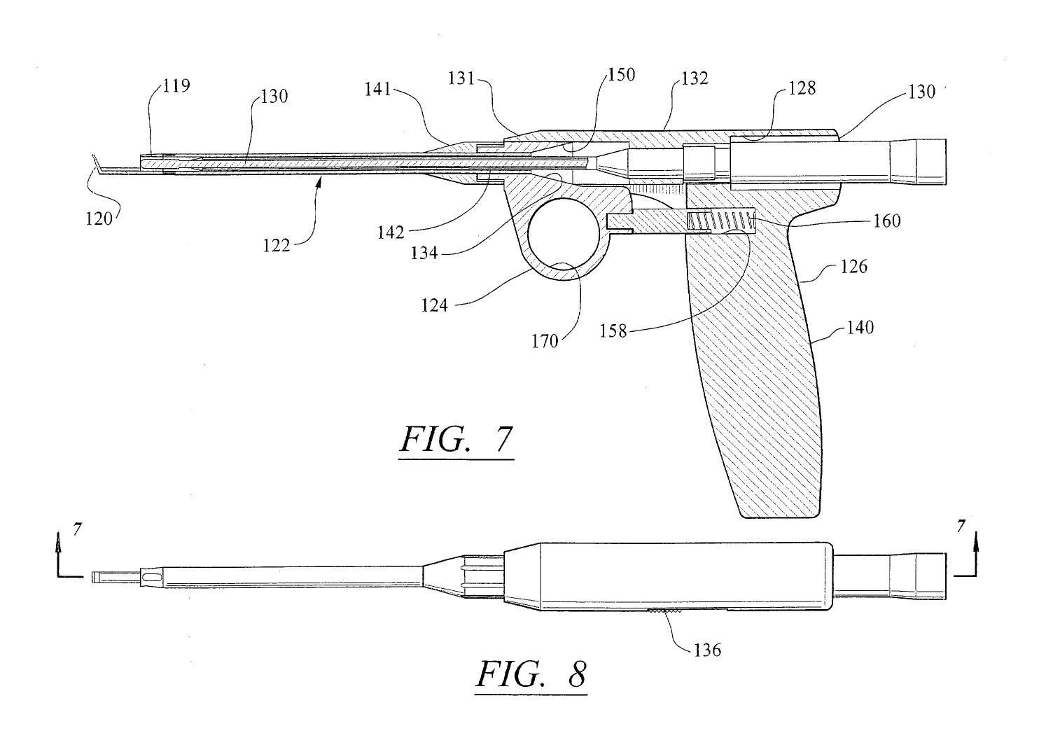

The next portion of the description relates to the other embodiment of this invention where the motor remains stationary and the foot plate is moved relative to the cutter. In this embodiment, as exemplified in FIGS. 7 and 8, the foot plate 120 attached to the foot plate support 122, similar in shape to the foot plate support depicted in FIG. 1, is attached to the trigger 124 so that when the trigger is retracted toward the handle 126 the foot plate 120 will move toward the drill bit 128 of cutter 130. Handle 126 is similar in its outer dimensions to the handle depicted in FIG. 1 and includes a through bore extending through the main portion 132 of handle 126 including a larger diameter portion 128 at the aft end 130 and stepped to smaller diameter portions toward the forward end 131 and increase again in the more forward portion 134. The motor 133, similar to the motor described in connection with FIG. 1 is installed in the rear portion of handle 126 and a suitable finger lock 136 movable in two positions locks and unlocks the motor 133. The cutter is removably attached to the motor in the customary fashion which is well known in the art. The handle 126, similarly shaped as a pistol handle, includes a hand held portion 140 extending downwardly from the main portion 132. Surrounding the foot plate support 122 is a conically shaped nose 141 that includes a well known attachment end that fits on the outer diameter of the reduced portion 142 adjacent the fore end of the trigger 124 that locks the foot plate support 142 to the trigger 124. The fore end of trigger 124 includes the torroidally shaped end portion 148 that extends rearwardly and defines a conically shaped bore 150. This shape assures that when the trigger is withdrawn, the foot plate will have sufficient room to allow the drill bit to move its intended distance and not lock up against the chuck end of the motor 133.

The trigger 124 is biased in the first position, which is the most forward position by the coil spring 156 mounted in bore 158 formed in the fore end of the hand-held portion 140 of handle 126. Coil spring 156 acts against the end face 160 of bore 158 and the piston 162 that includes tab 164 that fits into slot 166 of trigger 124.

In operation the surgeon holds the handle 128 as if it were a pistol and inserts a finger in the through opening 170. By squeezing the trigger 124 and handle 126 the trigger mechanism, just described, will move rearwardly bring the foot plate 120 closer to the drill bit 129 and with the motor turned on, allowing the surgeon to perform the procedure necessary to remove the intended bone of the patient. As is the case of the embodiment depicted in FIG. 1, the surgeon can move the instrument laterally from the local target so as to remove bone along the lateral direction. Also, since the PKS system utilizes a motor and drill bit and doesn't rely on brute force to remove bone, the foot plate can be designed into different shapes and angles so as to fit into areas of the body that were otherwise unavailable for this type of procedure.

Although this invention has been shown and described with respect to detailed embodiments thereof, it will be appreciated and understood by those skilled in the art that various changes in form and detail thereof may be made without departing from the spirit and scope of the claimed invention.

It is claimed:

1. A surgical instrument including a handle having a trigger mechanism, a motor mounted in said handle and an elongated tube connected to one end of said handle having an upstanding foot portion on the end remote from said handle, a cutter attached to said motor for rotary motion mounted in a central through-passage of the elongated tube, said trigger mechanism being movable in a rectilinear motion for positioning said motor and said cutter relative to and in close proximity to said foot portion, whereby a surgeon orients the bone and/or tissue intended to be cut between said foot portion and said cutter for performing the surgical procedure.

2. A surgical instrument as claimed in claim 1 wherein said handle is pistol-shaped and said motor is removable from said pistol-shaped handle, a rack and pinion gear mounted in said pistol-shaped handle between said trigger and said removable motor for positioning said motor/cutter relative to said foot.

3. A surgical instrument as claimed in claim 2 including a detent formed in said pistol-shaped handle, a shoulder on said motor for fitting into said detent for locking said motor in said pistol-shaped handle, means for pivoting said rack about said pinion gear for displacing said detent whereby said motor is unlocked and removable from said pistol-shaped handle.

5. A surgical instrument as claimed in claim 4 including a lever arm attached to said rack and adapted to being displaced by the operator to pivot said rack about said pinion gear.

6. A surgical instrument as claimed in claim 5 including a sleeve for receiving said motor, said sleeve having a boss extending therefrom, said lever being pivotally mounted to said boss, a spring mounted between said lever and said boss for biasing said lever in a locking position, whereby said motor being slidable into said sleeve and engaging said lever for displacing said lever for positioning said shoulder into said detent and said spring positioning said lever to the original position for locking said motor into said sleeve.

7. A surgical instrument as claimed in claim 7 including a coil spring biasing said handle in a predetermined position whereby said cutter is positioned away from said foot.

8. A surgical instrument as claimed in claim 1 whereby said foot is dimensioned and shaped for a given surgical procedure.

9. A surgical instrument including a handle having a trigger mechanism, a motor mounted in said handle and an elongated tube connected to one end of said handle having an upstanding foot portion on the end remote from said handle, a cutter attached to said motor for rotary motion mounted in a central through-passage of the elongated tube, said trigger mechanism being movable in a rectilinear motion for positioning said foot portion relative to and in close proximity to said cutter, whereby a surgeon orients the bone or tissue intended to be cut between said foot portion and said cutter for performing the surgical procedure.

9. A surgical instrument as claimed in claim 8 wherein said handle is pistol-shaped and said motor is removable from said pistol-shaped handle.

10. A surgical instrument as claimed in claim 8 whereby said foot is dimensioned and shaped for a given surgical procedure.

11. A surgical instrument as claimed in claim 9 including a piston having one end connected to said piston, a coil spring mounted in said handle having one end bearing against said piston to position said cutter in a given position whereby said cutter is furthest away from said foot.

12. A bone or tissue biting instrument including a handle having a trigger mechanism, a motor mounted in said handle, a nose sleeve removably attached to said motor, an elongated tube connected to one end of said handle having an upstanding foot portion on the end remote from said handle and concentric to and extending through a central through-passage in said nose sleeve, a cutter having a bit at the end thereof attached to said motor for rotary motion mounted in said central through-passage of said nose sleeve, said trigger mechanism being movable in a rectilinear motion for positioning said motor and said cutter relative to and in close proximity to said foot portion, whereby a surgeon orients the bone or tissue intended to be cut between said foot portion and said cutter for performing the surgical procedure.

13. A bone or tissue biting instrument as claimed in claim 12 wherein said handle is pistol-shaped and said motor is removable from said pistol-shaped handle, a rack and pinion gear mounted in said pistol-shaped handle between said trigger and said removable motor and operatively connected to said trigger for positioning said motor/cutter relative to said foot.

14. A bone or tissue biting instrument as claimed in claim 13 including a detent formed is said pistol-shaped handle, a shoulder on said motor for fitting into said detent for locking said motor in said pistol-shaped handle, means for pivoting said rack about said pinion gear for displacing said detent whereby said motor is unlocked and removable from said pistol-shaped handle.

15. A bone or tissue biting instrument as claimed in claim 14 including a lever arm attached to said rack and adapted to being displaced by the operator to pivot said rack about said pinion gear.

16. A bone or tissue biting instrument as claimed in claim 15 including a sleeve mounted in said handle for receiving said motor, said sleeve having a boss extending therefrom, said lever being pivotally mounted to said boss, a spring mounted between said lever and said boss for biasing said lever in a locking position, whereby said motor being slidable into said sleeve and engaging said lever for displacing said lever for positioning said shoulder into said detent and said spring positioning said lever back to the original position for locking said motor into said sleeve.

17. A bone or tissue biting instrument as claimed in claim 16 including a coil spring biasing said trigger in a predetermined position whereby said cutter is positioned away from said foot.

18. A surgical instrument as claimed in claim 12 whereby said foot is dimensioned and shaped for a given surgical procedure.

19. A surgical instrument including a handle having a trigger mechanism, a motor mounted in said handle and an elongated tube connected to one end of said handle having an upstanding foot portion on the end remote from said handle, a cutter attached to said motor for rotary motion mounted in a central through-passage of the elongated tube, said trigger mechanism being movable in a rectilinear motion for positioning said foot portion relative to and in close proximity to said cutter, whereby a surgeon orients the bone intended to be cut between said foot portion and said cutter for performing the surgical procedure.

20. A surgical instrument as claimed in claim 19 wherein said handle is pistol-shaped and said motor is removable from said pistol-shaped handle.

21. A surgical instrument as claimed in claim 20 whereby said foot is dimensioned and shaped for a given surgical procedure.

22. A surgical instrument as claimed in claim 21 including a piston having one end connected to said piston, a coil spring mounted in said handle having one end bearing against said piston to position said cutter in a given position whereby said cutter is furthest away from said foot.

23. A powered Kerrison Rongeur system including a handle having a trigger mechanism, a nose sleeve, a motor mounted in said handle and an elongated tube concentric to and mounted in a central through-passage in said nose sleeve and being connected to one end of said handle, said elongated tube having an upstanding foot portion on the end remote from said handle, a cutter having a cutting bit attached to said motor for rotary motion mounted in an elongated through passage of said nose sleeve, said trigger mechanism being movable in a rectilinear motion for positioning said foot portion relative to and in close proximity to said bit, whereby a surgeon orients the bone intended to be cut between said foot portion and said bit for performing the surgical procedure.

20. A powered Kerrison Rongeur system as claimed in claim 19 wherein said handle is pistol-shaped and said motor is removable from said pistol-shaped handle.

21. A powered Kerrison Rongeur system as claimed in claim 20 whereby said foot is dimensioned and shaped for a given surgical procedure.

22. A powered Kerrison Rongeur system as claimed in claim 21 including a piston having one end connected to said piston, a coil spring mounted in said handle having one end bearing against said piston to position said cutter in a given position whereby said cutter is furthest away from said foot.
